(19) United States
(12) Patent Application Publication
(10) Pub. No.: US 2014/0316449 A1
(43) Pub. Date: Oct. 23, 2014

(54) DEVICES, SYSTEMS AND METHODS FOR A QUICK LOAD GUIDE WIRE TOOL

(71) Applicant: Cardiovascular Systems, Inc., St. Paul, MN (US)

(72) Inventors: Preston Lee Grothe, Maple Grove, MN (US); Nicholas Ellering, Crystal, MN (US)

(73) Assignee: Cardiovascular Systems, Inc., St. Paul, MN (US)

(21) Appl. No.: 14/204,215

(22) Filed: Mar. 11, 2014

Related U.S. Application Data
(60) Provisional application No. 61/782,010, filed on Mar. 14, 2013.

(51) Int. Cl. A61B 17/3207

(52) U.S. Cl. CPC A61B 17/320758
USPC 606/159

(57) ABSTRACT

A system and method for loading a guide wire into a medical device is provided. The medical device comprises a drive shaft having a lumen and a distal end; and a guide wire loader having a distal end having a guide wire mating feature. In a preloaded state, at least a portion of the guide wire loader is disposed within the drive shaft lumen, and the distal end of the guide wire loader is disposed near the distal end the drive shaft. A guide wire has a loader mating feature on a proximal end of the guide wire that compliments the guide wire mating feature. To load the guide wire into the device, the loader mating feature of the guide wire is connected to the guide wire mating feature of the guide wire loader, and the guide wire loader shaft is moved axially in a proximal direction.

### CROSS-REFERENCE TO RELATED APPLICATIONS

This application claims priority to App. Ser. No. 61/782,010, entitled “Devices, Systems and Methods for a Quick Load Guide Wire Tool for Rotational Atherectomy,” filed Mar. 14, 2013.

### FIELD OF THE INVENTION

The present disclosure generally relates to devices and systems relating to rotational atherectomy devices. More specifically, a handheld guide wire loader is provided.

### DESCRIPTION OF THE RELATED ART

A variety of techniques and instruments have been developed for use in the removal or repair of tissue in arteries and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patentcy of the opened artery. For non-calcified lesions, balloon angioplasty most often is used alone to open the artery, and stents often are placed to maintain patentcy of the opened artery. Studies have shown, however, that a significant percentage of patients who have undergone balloon angioplasty and had a stent placed in an artery experience stent restenosis—i.e., blockage of the stent which most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patentcy of the artery.

Several kinds of rotational atherectomy devices have been developed for attempting to remove stenotic material. In one type of device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. When rotated at high speeds, the abrasive segment is capable of removing stenotic tissue from an artery. Though this atherectomy device possesses certain advantages over the Auth device due to its flexibility, it also is capable only of opening an artery to a diameter about equal to the diameter of the enlarged abrading surface of the drive shaft since the device is not eccentric in nature.

U.S. Pat. No. 6,494,890 (Shturman) discloses a known atherectomy device having a drive shaft with an enlarged eccentric section, wherein at least a segment of this enlarged section is covered with an abrasive material. When rotated at high speeds, the abrasive segment is capable of removing stenotic tissue from an artery. The device is capable of opening an artery to a diameter that is larger than the resting diameter of the enlarged eccentric section due, in part, to the orbital rotational motion during high speed operation. Since the enlarged eccentric section comprises drive shaft wires that are not bound together, the enlarged eccentric section of the drive shaft may flex during placement within the stenosis or during high speed operation. This flexion allows for a larger diameter opening during high speed operation, but may also provide less control than desired over the diameter of the artery actually abraded. In addition, some stenotic tissue may block the passageway so completely that the Shturman device cannot be placed therethrough. Since Shturman requires that the enlarged eccentric section of the drive shaft be placed within the stenotic tissue to achieve abrasion, it will be less effective in cases where the enlarged eccentric section is prevented from moving into the stenosis. The disclosure of U.S. Pat. No. 6,494,890 is hereby incorporated by reference in its entirety.

U.S. Pat. No. 5,681,336 (Clement) provides a known eccentric tissue removing burr with a coating of abrasive particles secured to a portion of its outer surface by a suitable binding material. This construction is limited, however because, as Clement explains at CoI. 3, lines 53-55, that the asymmetrical burr is rotated at “lower speeds than are used with high speed ablation devices, to compensate for heat or imbalance.” That is, given both the size and mass of the solid burr, it is infeasible to rotate the burr at the high speeds used during atherectomy procedures, i.e., 20,000-200,000 rpm. Essentially, the center of mass offset from the rotational axis of the drive shaft would result in development of significant centrifugal force, exerting too much pressure on the wall of the artery and creating too much heat and excessively large particles.

Generally atherectomy devices utilize a guidewire that extends distally from the distal end of the drive shaft to assist a practitioner in guiding the device through the patient's vasculature and to a desired location for removal of plaque or fatty tissue buildup. A guidewire, whether a new wire or a replacement wire, must be loaded into the atherectomy device such that it is controllable from a proximal end of the atherectomy device by the practitioner. Prior references that disclose methods and devices for loading a guide wire into a device, namely by coupling and/or connecting extension guide wires to the proximal end of the guide wire that is positioned within a patient's vasculature in order to effectively create a longer guidewire. These references include U.S. Pat. No. 5,404,888 (Kontos); U.S. Pat. No. 5,368,035 (Hamm); U.S. Pat. No. 5,290,232 (Johnson); U.S. Pat. No. 5,133,364 (Palermo); U.S. Pat. No. 5,271,415 (Foerster); U.S. Pat. No. 5,139,032 (Jahrmarkt); U.S. Pat. No. 5,113,872 (Jahrmarkt); U.S. Pat. No. 5,546,958 (Thorud); U.S. Pat. No. 6,217,526 (Frassica); U.S. Pat. Pub. No. 2011/0071435 (Shamay) and U.S. Pat. Pub. No. 2004/0039250 (Tholfsen); U.S. Pat. Pub. No. 2007/0299305 (Murakami); and U.S. 2009/0326449 (Wang), all of which are incorporated herein by reference. These prior art disclosures generally teach that by connecting these extension guide wires to a proximal end of the guide wire, the guide wire is pushed axially and distally to translate the distal end of the guide wire further into the patient's vasculature in preparation for a catheter exchange. Additionally, U.S. Pat. No. 8,267,873 (Yanuma), which is incorporated herein by reference, discloses a guide wire catheter that allows for movement of the guide wire in an axial direction and a circumferential direction. U.S. Pat. No. 5,813,405 (Montano, Jr.) discloses a snap-in connection assembly for an extension guide wire system for creation of a longer guide wire while the guide wire is already in the patient's vasculature. U.S. Pat. App. No. 2012/0253318 (Kimura) describes a device for applying a turning force to a guide wire that is already inserted into a patient's vasculature. U.S. Pat. No. 6,428,336 describes a device for connecting a guide wire to an interface cable, but again the guide wire is already inserted into the patient's vascular and the guide wire tool is not pre-loaded into the device.

There is a need in the art for improved devices for assisting with insertion of a guide wire into an atherectomy device or other medical devices, particularly in instances where the guide wire is not yet inserted into the patient's vasculature.

### BRIEF SUMMARY OF THE INVENTION

The present system is directed in various methods, devices and systems relating to loading a guide wire into a medical device. In particular, the methods, devices and systems are applicable to any over the wire interventional procedure, such as atherectomy. More specifically, a quick load guide wire tool is provided that is disposable and preloaded on the atherectomy system's drive shaft for assembly of the system, including the guide wire.

In at least one embodiment, a guide wire loading system having a preloaded state and a loaded state is provided. The system comprises a medical device having at least a handle portion; a catheter extending distally from the handle portion, the catheter having a catheter lumen; a drive shaft disposed within the catheter lumen, the drive shaft having a drive shaft lumen and a distal end; and a guide wire loader having a proximal end and a distal end having a guide wire mating feature, wherein in the preloaded state, at least a portion of the guide wire loader is disposed within the drive shaft lumen and the distal end of the guide wire loader is disposed near the distal end the drive shaft. The system further comprises a guide wire having a loader mating feature on a proximal end of the guide wire that compliments the guide wire mating feature at the distal end of the guide wire loader. During loading of the guide wire into the medical device from the preloaded state to the loaded state, the loader mating feature of the guide wire is connected to the guide wire mating feature of the guide wire loader, and the guide wire loader is moved axially in a proximal direction to load the guide wire into the drive shaft lumen.

In one embodiment, a medical device having a guide wire disposed within the device in a loaded state, the medical device comprises a handle portion; a catheter extending distally from the handle portion, the catheter having a catheter lumen; a drive shaft disposed within the catheter lumen; the drive shaft having a drive shaft lumen and a tip at a distal end of the drive shaft; and a guide wire loader having a proximal end and a distal end having a guide wire mating feature; wherein in a preloaded state, at least a portion of the guide wire loader is disposed within the drive shaft lumen and the distal end of the guide wire loader is disposed near the tip of the drive shaft.

In one embodiment, a method for loading a guide wire into the medical device is provided. connecting the guide wire mating feature of the guide wire loader with a loader mating feature on a proximal end of the guide wire; applying an axial force in a proximal direction to the guide wire loader. In at least one embodiment, the method further comprises releasing the guide wire mating feature from the loader mating feature.

### BRIEF DESCRIPTION OF THE DRAWINGS

FIG. 1 shows an embodiment of an atherectomy device with a guide wire.

FIG. 2 shows a cross-section of the drive shaft assembly of the atherectomy device, including the guide wire loader, in a preloaded state.

FIG. 3 shows a cross-section of the drive shaft assembly of FIG. 2 during loading of the guide wire.

FIG. 4A-4E shows perspective views of embodiments of the guide wire loader and the guide wire.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Although the invention is applicable to most medical devices incorporating a guide wire, particularly those devices for that include any over the wire interventional procedure, such as atherectomy devices, various embodiments of the present invention may be used with a rotational atherectomy system as described generally in U.S. Pat. No. 6,494,890, entitled “ECCENTRIC ROTATIONAL ATHERECTOMY DEVICE,” which is incorporated herein by reference. Additionally, the disclosure of the following co-owned patents or patent applications are herein incorporated by reference in their entireties: U.S. Pat. No. 6,295,712, entitled “ROTATIONAL ATHERECTOMY DEVICE”; U.S. Pat. No. 6,132,444, entitled “ECCENTRIC DRIVE SHAFT FOR ATHERECTOMY DEVICE AND METHOD FOR MANUFACTURE”; U.S. Pat. No. 6,638,288, entitled “ECCENTRIC DRIVE SHAFT FOR ATHERECTOMY DEVICE AND METHOD FOR MANUFACTURE”; U.S. Pat. No. 5,314,438, entitled “ABRASIVE DRIVE SHAFT DEVICE FOR ROTATIONAL ATHERECTOMY”; U.S. Pat. No. 6,217,595, entitled “ROTATIONAL ATHERECTOMY DEVICE”; U.S. Pat. No. 5,554,163, entitled “ATHERECTOMY DEVICE”; U.S. Pat. No. 7,507,245, entitled “ROTATIONAL ANGIOPLASTY DEVICE WITH ABRASIVE CROWN”; U.S. Pat. No. 6,129,734, entitled “ROTATIONAL ATHERECTOMY DEVICE WITH RADIALLY EXPANDABLE PRIME MOVER COUPLING”; U.S. Pat. No. 8,597,313, entitled “ECCENTRIC ABRADING HEAD FOR HIGH-SPEED ROTATIONAL ATHERECTOMY DEVICES”; U.S. Pat. No. 8,439,937, entitled “SYSTEM, APPARATUS AND METHOD FOR OPENING AN OCCLUDED LESION”; U.S. Pat. Pub. No. 2009/0299392, entitled “ECCENTRIC ABRADING ELEMENT FOR HIGH-SPEED ROTATIONAL ATHERECTOMY DEVICES”; U.S. Pat. Pub. No. 2010/0198239, entitled “MULTI-MATERIAL ABRADING HEAD FOR ATHERECTOMY DEVICES HAVING LATERALLY DISPLACED CENTER OF MASS”; U.S. Pat. Pub. No. 2010/0036402, entitled “ROTATIONAL ATHERECTOMY DEVICE WITH PRE-CURVED DRIVE SHAFT”; U.S. Pat. Pub. No. 2009/0299391, entitled “ECCENTRIC ABRADING AND CUTTING HEAD FOR HIGH-SPEED ROTATIONAL ATHERECTOMY DEVICES”; U.S. Pat. Pub. No. 2010/0100110, entitled “ECCENTRIC ABRADING AND CUTTING HEAD FOR HIGH-SPEED ROTATIONAL ATHERECTOMY DEVICES”; U.S. Design Pat. No. D610258, entitled “ROTATIONAL ATHERECTOMY ABRASIVE CROWN”; U.S. Design Pat. No. D6107102, entitled “ROTATIONAL ATHERECTOMY ABRASIVE CROWN”; U.S. Pat. Pub. No. 2009/0306689, entitled “BIDIRECTIONAL EXPANDABLE HEAD FOR ROTATIONAL ATHERECTOMY DEVICE”; U.S. Pat. Pub. No. 2010/0211088, entitled “ROTATIONAL ATHERECTOMY SEGMENTED ABRADING HEAD AND METHOD TO IMPROVE ABRADING EFFICIENCY”; U.S. Pat. Pub. No. 2013/0018398, entitled “ROTATIONAL ATHERECTOMY DEVICE WITH ELECTRIC MOTOR”; and U.S. Pat. No. 7,666,202, entitled “ORBITAL ATHERECTOMY DEVICE GUIDE WIRE DESIGN.” It is contemplated by this invention that the features of one or more of the embodiments of the present invention may be combined with one or more features of the embodiments of atherectomy devices described therein.

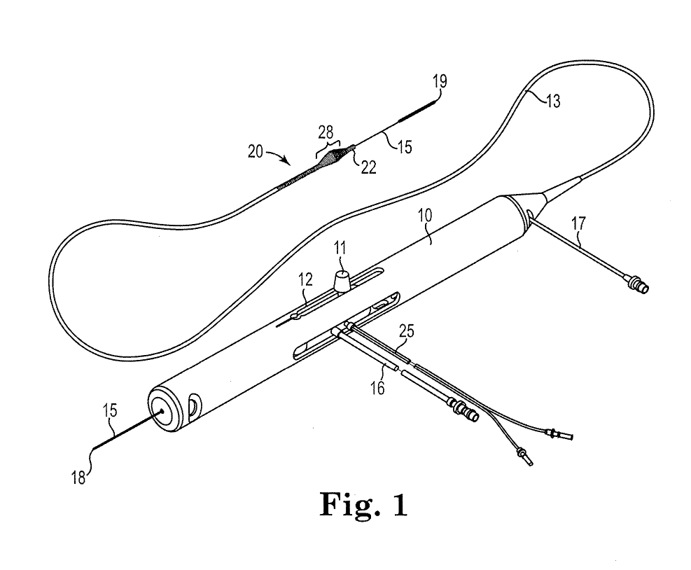

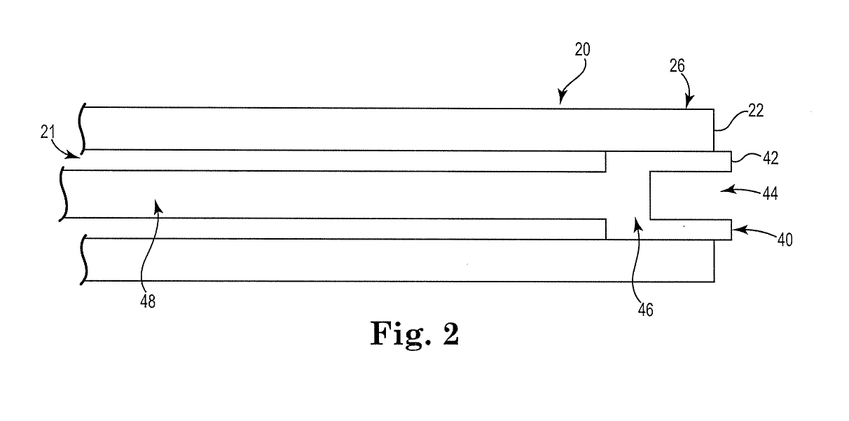

FIG. 1 illustrates an exemplary medical device in a loaded state (e.g. guide wire loaded into the drive shaft), specifically a rotational atherectomy device as described in U.S. Pat. No. 6,494,890, which is incorporated herein by reference. The device includes a handle portion 10; an elongated, flexible drive shaft 20 having an eccentric abrading head 28; and an elongated catheter 13 extending distally from the handle portion 10. The drive shaft 20 is constructed from helically coiled wire as is known in the art and the abrading head 28 is fixedly attached to the drive shaft 20. The drive shaft 20 has an inner lumen (21, shown in FIG. 2), permitting the drive shaft 20 to be advanced and rotated over a guide wire 15. The drive shaft 20 also has a distal end 22. Guide wire 15 has a proximal end 18 and a distal end 19. The catheter 13 has a lumen in which most of the length of the drive shaft 20 is disposed, except for the enlarged abrading head 28 and a section of the drive shaft 20 distal to the enlarged abrading head 28. A fluid supply line 17 may be provided for introducing a cooling and lubricating solution (typically saline or another biocompatible fluid) into the catheter 13.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20 (details regarding such handles and associated instrumentation are well known in the industry, and are described, e.g., in U.S. Pat. No. 5,314,407, issued to Auth, and incorporated herein by reference). The handle 10 also desirably includes a control knob 11 for advancing and retracting the turbine and drive shaft 20 with respect to the catheter 13 and the body of the handle.

As discussed above, in at least one embodiment, the eccentric abrading head 28 comprises an eccentric enlarged section of the drive shaft, or an eccentric solid crown, or an eccentric burr attached to the drive shaft. In some embodiments, the abrasive section 28 has a center of mass spaced radially from the rotational axis of the drive shaft 20, facilitating the ability of the device to open the stenotic lesion to a diameter substantially larger than the outer diameter of the abrasive section 28. This may be achieved by spacing the geometric center of the abrasive section 28, i.e., the eccentric enlarged diameter section of the drive shaft 20, or the eccentric solid abrading head or crown, or burr attached to the drive shaft 20, away from the rotational axis of the drive shaft 20. Alternatively, the center of mass of the abrading head 28 may be radially spaced from the drive shaft's rotational axis by providing an abrading head 28 that comprises a differential combination of materials, wherein one side of at least one of the abrading head 28 comprises a more massive or denser material than the other side, which creates eccentricity as defined herein. As those skilled in the art will recognize, creation of eccentricity as by differential use of materials within the structure of the abrading head 28, e.g., a center of mass offset from the drive shaft's rotational axis, is applicable to any embodiment of the abrading head 28 discussed herein, whether concentric, eccentric solid burr, partially hollow crown or abrading head or an enlarged section of the drive shaft, or the equivalent. When rotated at high rotational speeds, the drive shaft 20 stimulates orbital motion of the eccentric abrading head 28 to generate a cutting diameter that is greater than a diameter of the abrading head. In the present invention, the abrading head 28 may comprise a concentric profile or an eccentric profile. In some embodiments, the abrading head 28 may achieve orbital motion, generated by a positioning of the center of mass of the abrading head 28 radially offset from the rotational axis of the drive shaft, either by using different densities of materials and/or geometrically moving the center of mass of the abrading head 28 radially away from the drive shaft's center of mass. This “eccentricity” may be achieved in either a concentric or an eccentric geometric profile. The abrading head 28 may be an enlarged section of the drive shaft, a burr, or a contoured abrasive element and may comprise diamond coating. In other embodiments, the abrading head 28 may comprise a center of mass that is on the drive shaft's rotational axis.

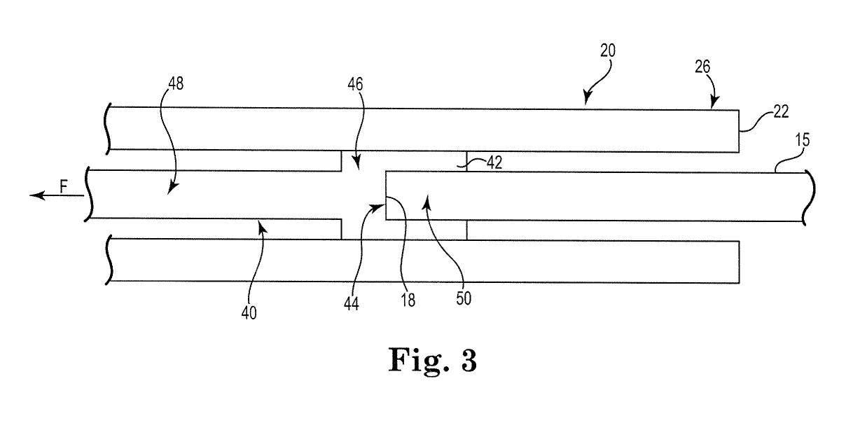

The present invention utilizes a loader 40, which is disposed within the drive shaft lumen 21 in a preloaded state of the device (in other words, no guide wire is disposed within the drive shaft lumen). FIG. 2 shows the device in a preloaded state and FIG. 3 shows the device during loading of the guide wire 15. As shown in FIG. 2, the loader 40 has a distal end 42 with a guide wire mating feature 44 disposed near the distal end 42 for engagement with a loader mating feature 50 on the guide wire 15 (shown in FIG. 3). Loader 40 may be engaged with guide wire 15 through a compression fit, an interference fit, or other frictional engagement. Additionally loader 40 may be engaged with guide wire 15 in a locking engagement (such as, e.g., a screw and thread arrangement). In other embodiments, loader 40 may be engaged with the guide wire 15 via magnetic forces. In still other embodiments, loader 40 may be engaged with the guide wire 15 with adhesives or hook and loop fasteners. Examples of guide wire mating features and related loader mating features will be discussed further below. In some embodiments, such as the embodiments shown in FIGS. 2-3, the loader 40 has a tip 46 near distal end 42 of the loader 40 and a shaft portion 48 extending proximally from the tip 46. The tip 46 may have a diameter greater than a diameter of the shaft portion.

In at least one embodiment, as shown in FIG. 2, in the preloaded state, the distal end 42 of the loader 40 is positioned distally from the distal end 22 of the drive shaft to aid in the facilitation of connecting the loader 40 with the guide wire 15. In other embodiments, the distal end 42 of the loader 40 may be positioned at the distal end 22 of the drive shaft 20 in the preloaded state. In some embodiments, the distal end 42 of the loader 40 may be positioned proximally from the distal end 22 of the drive shaft 40, so long as the guidewire 15 is capable of engagement with the loader 40. In some embodiments, the drive shaft 20 may have a tip section 26, which includes the distal end 22, and in such embodiments, the drive shaft may have a bushing to help with rotational movement of the loader 40 relative to the drive shaft 22 if needed.

In some embodiments, in the preloaded state, a proximal portion of the loader 40 extends proximally from the handle 10 of the device. The proximal portion may have a gripping feature on an outer surface of the proximal portion to help facilitate axial movement of the handle 10 by a user. In some embodiments, the handle 10 may include a grip that is attached to the loader 40 so that when the grip is pulled proximally away from the handle assembly, the loader 40 and guide wire

To load the guide wire into the device, the loader mating feature 50 of the guide wire 15 is first engaged with the guide wire mating feature 44 of the loader 40, as shown in FIG. 3. An axial force F is then applied in a proximal direction to the loader 40, and the guide wire 15 is pulled axially in a proximal direction through the drive shaft lumen 21. In some embodiments, once the guide wire 15 is pulled proximally from the proximal end of the handle 10, the loader 40 may be disengaged with the guide wire 15. In at least some embodiments, the outer surface of the loader 40 comprises a lubricious coating, which may facilitate smooth movement of the loader 40 through the drive shaft lumen 21.

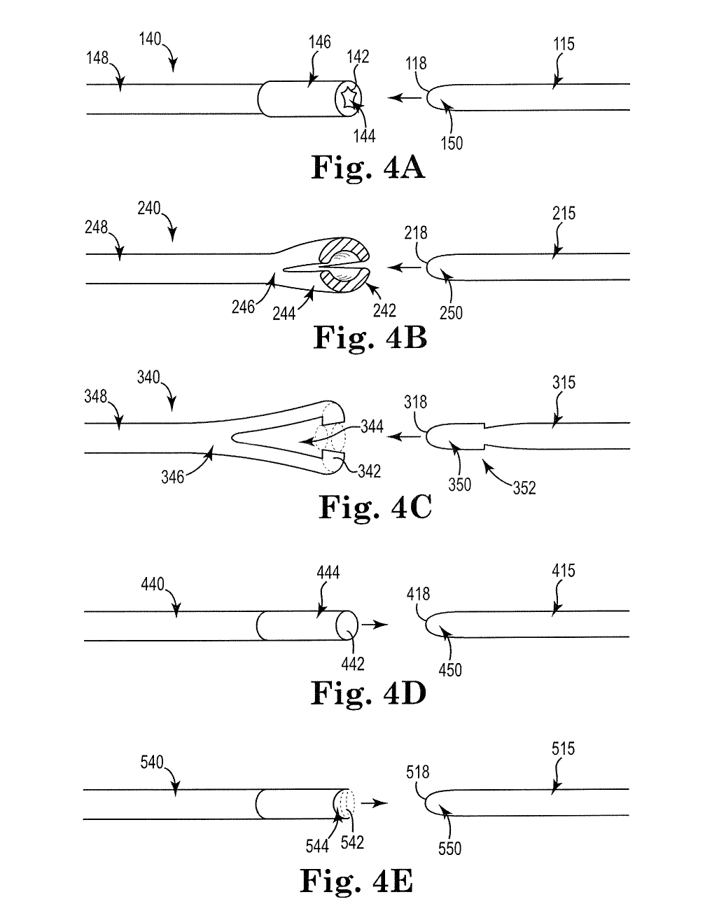

FIGS. 4A-4E provide exemplary combinations of compatible guide wire mating features and loader mating features for use with the invention of the present disclosure. Other suitable combinations of engageable and compatible guide wire mating features and loading mating features are within the contemplation of this invention. As discussed above, frictional engagement mechanisms, locking engagement mechanisms, magnetic forces, and adhesives may be used to connect the guide wire with the loader. Desirably, the mating features prevent relative axial and rotational movement between the guide wire and the loader.

FIG. 4A shows one embodiment of guide wire 115 and loader 140 with tip 146 and shaft 148, where the guide wire mating feature 144 is a socket disposed at a distal end 142 of the loader 140 and extends axially into tip 146. The loader mating feature 150 is shaped for retention by the socket. For example, if the profile of the socket is a hexagon, the outer surface of loader mating feature 150 is likewise shaped like a hexagon.

FIG. 4B shows one embodiment of guide wire 215 and loader 240 with tip 246 and shaft 248, where the guide wire mating feature 244 is a collet disposed at a distal end 242 of the loader 240. The loader mating feature 250 is shaped for retention by the collet and may be of any suitable configuration.

FIG. 4C shows one embodiment of guide wire 315 and loader 340 with tip 346 and shaft 348, where the guide wire mating feature 344 is a gripping claw. The loader mating feature 350 on guide wire 315 is shaped for retention by the gripping claw and may be of any suitable configuration. As shown in FIG. 4C, loader mating feature 350 is a bulbous tip 351 near proximal end 318 with a narrow neck portion 352. When engaged, the claw grips the guide wire 315 at the narrow neck portion 352 and the bulbous tip 351 rests between the upper and lower portions of the claw.

FIG. 4D shows one embodiment of guide wire 415 and loader 440 where the guide wire 415 and loader 440 are connected via a magnetic force. In the embodiment shown, the guide wire mating feature is a magnet disposed within tip 446 near end 442, and the loader mating feature 450 on guide wire 415 comprises a ferromagnetic material. In another embodiment, the loader mating feature 450 may be a magnet disposed within the guide wire near end 418, and the guide wire mating feature 444 comprises a ferromagnetic material.

FIG. 4E shows another embodiment of guide wire 515 and loader 540 where the two elements are connected via an adhesive. In one embodiment, the guide wire mating feature 544 is an adhesive disposed on the end 542 of the loader 540, and the loader mating feature 550 be the proximal end 518. The loader mating feature 550 may or may not have a coating or adhesive that facilitates adhesion between the guide wire 515 and the loader 540. In at least one embodiment, once adhered, the guide wire 515 and loader 540 may still later be separated once the guide wire 515 is loaded into the drive shaft. In the embodiment shown in FIG. 4E, guide wire mating feature 544 may also include a concave surface at the end 542 that mates with the shape of the proximal end 518 of the guide wire 515, to further facilitate the engagement between the guide wire 515 and the loader 540.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A guide wire loading system having a preloaded state and a loaded state comprising:
a medical device comprising:
a handle portion;
a catheter extending distally from the handle portion, the catheter having a catheter lumen;
a drive shaft disposed within the catheter lumen;
the drive shaft having a drive shaft lumen and a distal end; and
a guide wire loader having a proximal end and a distal end having a guide wire mating feature, wherein in the preloaded state, at least a portion of the guide wire loader is disposed within the drive shaft lumen and the distal end of the guide wire loader is disposed near the distal end of the drive shaft; and
a guide wire having a loader mating feature on a proximal end that compliments the guide wire mating feature at the distal end of the guide wire loader;
wherein during loading of the guide wire into the medical device from the preloaded state to the loaded state, the loader mating feature of the guide wire is connected to the guide wire mating feature of the guide wire loader, and the guide wire loader is moved axially in a proximal direction to load the guide wire into the drive shaft lumen.

2. The system of claim 1, wherein the medical device is a rotational atherectomy device.

3. The system of claim 1, wherein in the preloaded state, the distal end of the guide wire loader is positioned proximally of the distal end of the drive shaft.

4. The system of claim 1, wherein in the preloaded state, the distal end of the guide wire loader is positioned distally from the distal end of the drive shaft.

5. The system of claim 1, wherein in the preloaded state, the distal end of the loader is positioned at the distal end of the drive shaft.

6. The system of claim 1, wherein the drive shaft has a tip.

7. The system of claim 6, wherein the drive shaft has a bushing at the tip.

8. The system of claim 1, wherein the guide wire mating feature is a socket and the loader mating feature is shaped for retention by the socket.

9. The system of claim 1, wherein the guide wire mating feature is a collet and the loader mating feature is shaped for retention by the collet.

10. The system of claim 1, wherein the guide wire mating feature is a gripping claw and the loader mating feature is shaped to mate with the claw.

11. The system of claim 1, wherein the guide wire mating feature comprises a magnet and the loader mating feature comprises a ferromagnetic material.

12. The system of claim 1, wherein the loader mating feature comprises a magnet and the guide wire mating feature comprises a ferromagnetic material.

13. The system of claim 1, wherein at least one of the guide wire mating feature and the loader mating feature comprises an adhesive.

14. The system of claim 1, wherein the handle portion further comprises a grip connected to the guide loader.

15. A medical device having a guide wire disposed within the device in a loaded state, the medical device comprising:
a handle portion;
a catheter extending distally from the handle portion, the catheter having a catheter lumen;
a drive shaft disposed within the catheter lumen;
the drive shaft having a drive shaft lumen and a tip at a distal end of the drive shaft; and
a guide wire loader having a proximal end and a distal end having a guide wire mating feature;
wherein in a preloaded state, at least a portion of the guide wire loader is disposed within the drive shaft lumen and the distal end of the guide wire loader is disposed near the tip of the drive shaft.

16. The device of claim 15, wherein during loading of the guide wire into the medical device from the preloaded state to the loaded state, the loader mating feature of the guide wire is connected to the guide wire mating feature of the guide wire loading shaft, and the guide wire loading shaft is moved axially in a proximal direction to load the guide wire into the drive shaft lumen.

17. The system of claim 16, wherein the handle portion further comprises a grip connected to the guide loader.

18. The system of claim 15, wherein the medical device is a rotational atherectomy device.

19. The system of claim 15, wherein in the preloaded state, the distal end of the guide wire loader is positioned proximally of the distal end of the drive shaft.

20. The system of claim 15, wherein in the preloaded state, the distal end of the guide wire loader is positioned distally from the distal end of the drive shaft.

21. The system of claim 15, wherein in the preloaded state, the distal end of the loader is positioned at the distal end of the drive shaft.

22. The system of claim 15, wherein the drive shaft has a tip.

23. The system of claim 6, wherein the drive shaft has a bushing at the tip.

24. The system of claim 15, wherein the guide wire mating feature is a socket.

25. The system of claim 15, wherein the guide wire mating feature is a collet and the loader mating feature is shaped for retention by the collet.

26. The system of claim 15, wherein the guide wire mating feature is a gripping claw.

27. The system of claim 15, wherein the guide wire mating feature comprises a magnet.

28. The system of claim 15, wherein the guide wire mating feature comprises a ferromagnetic material.

29. The system of claim 15, wherein the guide wire mating feature comprises an adhesive.

30. A method of loading a guide wire into a medical device, the method comprising:
providing a medical device comprising a handle portion; a catheter extending distally from the handle portion, the catheter having a catheter lumen; a drive shaft disposed within the catheter lumen; the drive shaft having a drive shaft lumen and a tip at a distal end of the drive shaft; and a guide wire loader disposed within the drive shaft lumen, the guide wire loader having a proximal end and a distal end having a guide wire mating feature;
connecting the guide wire mating feature of the guide wire loader with a loader mating feature on a proximal end of the guide wire;
applying an axial force in a proximal direction to the guide wire loader.

31. The method of claim 30, further comprising releasing the guide wire mating feature from the loader mating feature.

32. The system of claim 30, wherein the medical device is a rotational atherectomy device.

33. The system of claim 30, wherein in the preloaded state, the distal end of the guide wire loader is positioned proximally of the distal end of the drive shaft.

34. The system of claim 30, wherein in the preloaded state, the distal end of the guide wire loader is positioned distally from the distal end of the drive shaft.

35. The system of claim 30, wherein in the preloaded state, the distal end of the loader is positioned at the distal end of the drive shaft.

36. The system of claim 30, wherein the drive shaft has a tip.

37. The system of claim 36, wherein the drive shaft has a bushing at the tip.

38. The system of claim 30, wherein the guide wire mating feature is a socket and the loader mating feature is shaped for retention by the socket.

39. The system of claim 30, wherein the guide wire mating feature is a collet and the loader mating feature is shaped for retention by the collet.

40. The system of claim 30, wherein the guide wire mating feature is a gripping claw and the loader mating feature is shaped to mate with the claw.

41. The system of claim 30, wherein the guide wire mating feature comprises a magnet and the loader mating feature comprises a ferromagnetic material.

42. The system of claim 30, wherein the loader mating feature comprises a magnet and the guide wire mating feature comprises a ferromagnetic material.

43. The system of claim 30, wherein at least one of the guide wire mating feature and the loader mating feature comprises an adhesive.

44. The system of claim 30, wherein the handle portion further comprises a grip connected to the guide loader.
